(19) United States
(12) Patent Application Publication
(10) Pub. No.: US 2026/0198786 A1
(43) Pub. Date: Jul. 16, 2026

(54) METHOD AND SYSTEM FOR IDENTIFYING CARDIAC PARAMETERS

(71) Applicant: CARDIACSENSE LTD., Caesarea (IL)

(72) Inventor: Eldad SHEMESH, Binyamina (IL)

(21) Appl. No.: 19/132,985

(22) PCT Filed: Nov. 26, 2023
(86) PCT No.: PCT/IL2023/051213

§ 371 Date: May 27, 2025

(30) Foreign Application Priority Data
Nov. 27, 2022 (IL) 298601

(51) Int. Cl. A61B 5/0205; A61B 5/00; A61B 5/024; A61B 5/08; A61B 5/33

(52) U.S. Cl. CPC A61B 5/0205; A61B 5/33; A61B 5/681; A61B 5/746; A61B 5/02416; A61B 5/0816

(57) ABSTRACT

The present disclosure provides a unique solution that allows to identify in real-time one or more physiological parameters of a subject that is continuously being monitored with at least an optical sensor, such as a PPG sensor, that provides a continuous signal indicative of blood volume changes in the microvascular bed of tissue of the subject. The solution provided by the present disclosure is allowed due to analysis of the signal and deriving from it the characterizations of single for each identified signal profile strokes of the heart that are identified by parameters in the signal profile. An analysis of these parameters yields results indicative of the stroke volume of each identified stroke. In a first solution or any other known deviation calculation of a series of measurements, it can be determined whether the subject is having a cardiac irregularity or not. Upon identifying a cardiac irregularity, an alert is outputted indicating to the subject, a caregiver, a user or any relevant person that is related to the subject, that a cardiac irregularity was identified. In a second solution of the present disclosure, the variation profile of the stroke volumes of the subject irregular strokes above a defined number threshold is determined, and signatures of the beginning and/or ending of a single respiratory cycle are identified to allow classification of single respiratory cycle of the subject. Based on that, a calculation of the number of single respiratory cycles in a selected period of time is performed to determine the respiratory rate of the subject.

### TECHNOLOGICAL FIELD

The present disclosure is in the field of medical monitoring systems, in particular wearable medical systems such as a medical watch.

### BACKGROUND ART

References considered to be relevant as background to the presently disclosed subject matter are listed below:
WO 2022/024113

Acknowledgement of the above references herein is not to be inferred as meaning that these are in any way relevant to the patentability of the presently disclosed subject matter.

### GENERAL DESCRIPTION

The present disclosure provides a unique solution that allows to identify in real-time one or more physiological parameters, including cardiovascular parameters, of a subject that is continuously being monitored with at least an optical sensor, such as a PPG sensor, that provides a continuous signal indicative of blood volume changes in the microvascular bed of tissue of the subject. The solution provided by the present disclosure is allowed due to analysis of the signal and deriving from it the characterizations of single strokes of the heart that are identified by parameters in the signal profile. An analysis of these parameters yields results indicative to or correlative of the stroke volume of each identified stroke.

In a first solution of the present disclosure, by calculating the standard deviation, or any other known deviation calculation of a series of measurements, it can be determined whether the subject is having a cardiac irregularity or not. Upon identifying a cardiac irregularity, an alert is outputted indicating to the subject, a caregiver, a user or any relevant person that is related to the subject, that a cardiac irregularity was identified.

In a second solution of the present disclosure, the variation profile of the stroke volumes of the subject is determined, and signatures of the beginning and/or ending of a single respiratory cycle are identified to allow classification of single respiratory cycle of the subject. Based on that, a calculation of the number of single respiratory cycles in a selected period of time is performed to determine the respiratory rate of the subject.

Therefore, an aspect of the present disclosure provides a method for real-time identification of physiological parameters in a subject. The method comprising receiving a temporal optical signal indicative of blood volume changes in the microvascular bed of tissue of the subject; identifying, in one or more selected time periods of said temporal optical signal, signal profiles indicative of single strokes; based on the optical signal profile, determining or calculating a stroke volume parameter. The term “stroke volume parameter” is used herein to denote a parameter that is indicative of or correlative to the stroke volume of identified signal strokes and that is determined based on parameters of the optical signal profile. The term “stroke volume” may be used interchangeably with the term “stroke volume parameter”, it is being understood that while the stroke volume parameter by some embodiments may be the stroke volume, in other embodiments the stroke volume parameter may a value indicative of the stroke volume, for example an area under the curve of the PPG signal, and so forth. Thus, the term “stroke volume” when used in connection with a determined parameter, should be understood to denote a “stroke volume parameter”.

The method further comprises at least one of the following:
(1) for identified single strokes, calculating a deviation parameter, and generating deviation parameter data indicative of said one or more cardiovascular parameters. The deviation parameter indicates the irregularity of the cardiovascular activity of the subject and therefore can indicate that the subject is having cardiac arrhythmia; and
(2) profiling the temporal variation of the determined stroke volume parameters to identify profile signatures indicative of a single respiratory cycle of the subject, calculating the respiration cycles over the selected time period to extract the respiration rate of the subject, and generating or outputting respiration data indicative of the calculated respiration rate of the subject.

It is to be noted that any combination of the embodiments described herein with respect to any aspect of this present disclosure is applicable also to other aspects. In other words, any aspect of the present disclosure can be carried out by any combination of the described embodiments.

In some embodiments of the method, the temporal optical signal is a photoplethysmogram (PPG) signal.

In some embodiments of the method, if the deviation parameter is above a defined deviation parameter threshold, which can be determined according to the cardiac irregularly that is sought, outputting an alert generating irregular cardiac activity data based thereon. The method may comprise outputting said irregular cardiac activity data to create an alarm that can be identified by the subject or any other caregiver.

In some embodiments of the method, said calculating comprises calculating a number of irregular strokes, irregular strokes being defined by a deviation in their determined stroke volume that is greater than a defined deviation threshold from a subject's reference stroke volume, wherein the number of irregular strokes is the deviation parameter.

In some embodiments of the method, said calculating comprises comparing identified stroke volumes to a reference parameter to obtain said deviation parameter.

In some embodiments of the method, the reference parameter is a subject's reference stroke volume, and the deviation parameter is the number of irregular strokes, wherein irregular strokes are defined by a deviation in their determined stroke volume that is greater than a defined deviation threshold from the subject's reference stroke volume.

In some embodiments of the method, the deviation parameter is indicative of the standard deviation of the stroke volume of identified signal strokes.

In some embodiments of the method, said identifying comprises identifying, in one or more selected time periods of said temporal optical signal, time intervals of single strokes.

In some embodiments of the method, said selected time periods are in the range of between 5, 10, 15, 20, 25, 30 to 50, 100, 150, 200 or more seconds. Namely, the analysis of the deviation of the stroke volumes is performed on a signal being collected along this range of time period.

In some embodiments of the method, said determining comprises determining or calculating the stroke volume parameter indicative of the stroke volume of identified signal strokes in said identified respective time interval.

In some embodiments of the method, said receiving comprises sensing said temporal optical signal from the subject.

In some embodiments of the method, the temporal optical signal is measured from a wrist of the subject, namely by illuminating the arterioles through the skin in this area. This can be performed by a medical watch worn by the subject and continuously sensing a PPG signal from the wrist of the subject.

In some embodiments of the method, said identifying comprises identifying the local minimums of the temporal optical signal and define each two adjacent local minimums as a time interval of a single stroke.

In some embodiments of the method, said identifying comprises identifying local maximums of the temporal optical signal and define the stroke interval as a certain, and maybe predetermined, time range that includes time before and after the respective local maximum.

In some embodiments of the method, said determining comprises integrating the signal in each identified time interval, wherein the integration is performed with respect to a selected base line reference.

In some embodiments of the method, said base line is defined as the line connecting two local minimums defining said time interval.

In some embodiments, the method comprising, prior to calculating, comparing each stroke volume to the reference stroke volume to derive a deviation degree, either in percentage or absolute difference of a parameter, of the respective stroke volume from the reference stroke volume.

In some embodiments of the method, the reference stroke volume is either predetermined or determined based on a selected group of the determined stroke volumes.

In some embodiments of the method, the reference stroke volume is determined based on a mean value, average or any combination thereof of said selected group.

In some embodiments of the method, said deviation threshold is defined as a deviation of at least 20%, or at least 10%, 30%, 40%, 50%, 60%, 70%, 80%, 90% 100%, 200%, 300%, 400%, 500%, 600%, 700%, 800%, 900% or at times 1000% from the value of the reference stroke volume.

In some embodiments of the method, said deviation threshold is defined as a deviation of at least 0.5, 0.75, 1, 1.25, 1.5, 2, or more of the standard deviation of all stroke volumes in said selected time period.

In some embodiments, the method comprising receiving temporal electrocardiogramaignal of the subject correlated with said temporal optical signal. Said identifying comprises initially identifying the time intervals in said temporal ECG signal and using time stamps of the identified intervals in said temporal ECG signal in said temporal optical signal to identify the time interval in said temporal optical signal that are associated with single strokes.

In some embodiments of the method, said alert is one of: visual signal, electronic signal, e.g. signal to a medical watch to be presented on a display thereof or a signal being sent to a caregiver.

In some embodiments, the method further comprising determining a respiration rate of the subject based on the number of identified stroke volumes over said selected time period.

In some embodiments of the method, said profiling comprises identifying two consecutive local minimums and/or two consecutive maximums of the variation profile of determined stroke volumes to thereby identify a single respiration cycle.

In some embodiments, the method comprising (1) and not (2), namely only comprising calculating a number of irregular strokes, wherein irregular strokes are defined by a deviation in their determined volume that is greater than a defined deviation threshold from a subject's reference stroke volume, and if the number of irregular strokes in at least one of said one or more selected time periods is above a defined number threshold, which can be determined according to the cardiac irregularly that is sought), outputting an alert.

In some embodiments, the method comprising (1) and not (2), namely only comprising profiling the variation of the determined stroke volumes to identify profile signatures indicative of a single respiratory cycle of the subject, calculating the respiration cycles over the selected time period to extract the respiration rate of the subject, and outputting respiration data indicative of the calculated respiration rate of the subject.

In some embodiments, the method further comprising sensing artifact signals in said temporal optical signal. The method further comprising cancelling stroke volumes and/or parts of the temporal optical signal that are associated with one or more artifact signals. This process reduces the false alarms of cardiac irregularities. The sensing of the artifact signals can be performed by a displacement sensor configured to sense artifact movements of the skin portion from which the optical signal is measured.

Yet another aspect of the present disclosure provides a system for real-time identification of cardiovascular parameters in a subject. The system comprising a processing circuitry that is configured for:
(i) receiving a temporal optical signal indicative of blood volume changes in the microvascular bed of tissue of the subject;
(ii) identifying, in one or more selected time periods of said temporal optical signal, signal profiles indicative of single strokes;
(iii) based on the optical signal's profile, determining a stroke volume parameter indicative of or correlative to the stroke volume of identified signal strokes;
(iv) wherein the processing circuitry is configured for performing at least one of the following:
   (1) for identified signal strokes, calculating a deviation parameter, which can be determined according to the cardiac irregularly that is sought, and generating deviation parameter data indicative of said one or more cardiovascular parameters; and
   (2) profiling the temporal variation of the determined stroke volume parameters to identify profile signatures indicative of a single respiratory cycle of the subject, calculating the respiration cycles over the selected time period to extract the respiration rate of the subject, and generating and optionally outputting respiration data indicative of the calculated respiration rate of the subject.

The processing circuitry may be coupled to one or more memory utilities storing instructions for execution by the processing circuitry configuring the processing circuitry to carry out the above. Alternatively, the processing circuitry may have some or all the above processing instructions embedded in the circuitry.

In some embodiments of the system, the temporal optical signal is a photoplethysmogram (PPG) signal.

In some embodiments of the system, if the deviation parameter is above a defined deviation parameter threshold, the processing circuitry is configured for generating irregular cardiac activity data based thereon. The processing circuitry may further be configured for outputting said irregular cardiac activity data to create an alarm that can be identified by the subject or any other caregiver.

In some embodiments of the system, said calculating comprises calculating a number of irregular strokes, irregular strokes being defined by a deviation in their determined stroke volume that is greater than a defined deviation threshold from a subject's reference stroke volume, wherein the number of irregular strokes is the deviation parameter.

In some embodiments of the system, said calculating comprises comparing identified stroke volumes to a reference parameter to obtain said deviation parameter.

In some embodiments of the system, the reference parameter is a subject's reference stroke volume, and the deviation parameter is the number of irregular strokes, wherein irregular strokes are defined by a deviation in their determined stroke volume that is greater than a defined deviation threshold from the subject's reference stroke volume.

In some embodiments of the system, the deviation parameter is indicative of the standard deviation of the stroke volume of identified signal strokes.

In some embodiments of the system, said identifying comprises identifying, in one or more selected time periods of said temporal optical signal, time intervals of single strokes.

In some embodiments of the system, said selected time periods are in the range of between 5, 10, 15, 20, 25, 30 to 50, 100, 150, 200 or more seconds.

In some embodiments of the system, said determining comprises determining or calculating the stroke volume or a parameter indicative of the stroke volume of identified signal strokes in said identified respective time interval.

In some embodiments of the system, wherein said determining comprises integrating the signal in each identified time interval, wherein the integration is performed with respect to a selected base line reference.

In some embodiments of the system, wherein said base line is defined as the line connecting two local minimums defining said time interval.

In some embodiments, the system further comprising an optical sensor configured to sense said temporal optical signal and transmit it to the processing circuitry.

In some embodiments of the system, said optical sensor is a PPG sensor.

In some embodiments of the system, the optical sensor is configured to illuminate a skin portion of the wrist of the subject to obtain said temporal optical signal, namely by illuminating the arterioles through the skin in this area. This can be performed by a medical watch worn by the subject and continuously sensing a PPG signal from the wrist of the subject.

In some embodiments of the system, said identifying comprises identifying the local minimums of the temporal optical signal and define each two adjacent local minimums as a time interval of a single stroke.

In some embodiments of the system, said identifying comprises identifying local maximums of the temporal optical signal and define the stroke interval as a certain, and maybe predetermined, time range that includes time before and after the respective local maximum.

In some embodiments of the system, the processing circuitry is further configured for comparing each stroke volume to the reference stroke volume to derive a deviation degree, either in percentage or absolute difference of a parameter, of the respective stroke volume from the reference stroke volume, the deviation threshold is defined by a certain deviation degree, i.e., if the deviation degree is above a certain threshold, the stroke volume is considered to be irregular and counted as one.

In some embodiments of the system, the reference stroke volume is either predetermined or determined based on a selected group of the determined stroke volumes.

In some embodiments of the system, the reference stroke volume is determined based on a mean value, average or any combination thereof of said selected group.

In some embodiments of the system, said deviation threshold is defined as at least one of the following: (i) a deviation of at least 20% or at least 10%, 30%, 40%, 50%, 60%, 70%, 80%, 90%, 100%, 200%, 300%, 400%, 500%, 600%, 700%, 800%, 900% or at times 1000%, from the reference stroke volume, or (ii) a deviation of at least 0.5, 0.75, 1, 1.25, 1.5, 2, or more of the standard deviation of all the determined stroke volumes in a selected period of time.

In some embodiments, the system further comprising an ECG sensor configured to sense a temporal ECG signal of the subject and transmit it to the processing circuitry. The processing circuitry is further configured for correlating the ECG signal with said temporal optical signal, and wherein said identifying comprises initially identifying the time intervals in said temporal ECG signal and using time stamps of the identified intervals in said temporal ECG signal in said temporal optical signal to identify the time interval in said temporal optical signal.

In some embodiments, the system further comprising an alerting unit configured to execute an alarm in response to receiving said alert signal.

In some embodiments of the system, said alert signal is either visual or audible.

In some embodiments of the system, the alerting unit comprises a display and/or speaker for displaying and/or sounding an alert to the user.

In some embodiments of the system, the processing circuitry is further configured for determining a respiration rate of the subject based on the number of identified stroke volumes over said selected time period.

In some embodiments of the system, said profiling comprises identifying two consecutive local minimums and/or two consecutive maximums of the variation profile of determined stroke volumes to thereby identify a single respiration cycle.

In some embodiments of the system, the processing circuitry is only configured for performing (1) and not (2), namely only configured for calculating a number of irregular strokes, wherein irregular strokes are defined by a deviation in their determined volume that is greater than a defined deviation threshold from a subject's reference stroke volume, and if the number of irregular strokes in at least one of said one or more selected time periods is above a defined number threshold, which can be determined according to the cardiac irregularly that is sought), outputting an alert.

In some embodiments of the system, the processing circuitry is only configured for performing (1) and not (2), namely only comprising profiling the variation of the determined stroke volumes to identify profile signatures indicative of a single respiratory cycle of the subject, calculating the respiration cycles over the selected time period to extract the respiration rate of the subject, and outputting respiration data indicative of the calculated respiration rate of the subject.

In some embodiments, the system further comprising an artifact sensor configured for sensing artifact signals in said temporal optical signal. The processing circuitry is further configured to cancel stroke volumes and/or parts of the temporal optical signal that are associated with one or more artifact signals. This process reduces the false alarms of cardiac irregularities.

The artifact sensor can be based on a displacement sensor configured to sense artifact movements of the skin portion from which the optical signal is measured.

Yet another aspect of the present disclosure provides a medical watch comprising the system of any one of the above embodiments or any combination thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are flow charts exemplifying different embodiments of the method according to an aspect of the present disclosure.

FIGS. 2A-2C are block diagrams exemplifying different embodiments of the system according to an aspect of the present disclosure.

FIGS. 3A-3C are examples of optical signals derived from different patients and the analysis these signals undergo according to the solution provided by the present disclosure.

FIG. 4 is an example of a report that is outputted by the system of the present disclosure.

FIGS. 5A-5B is an example of calculated stroke volumes of a subject over time and the determined variation profile of said stroke volumes over time.

### DETAILED DESCRIPTION

The following figures are provided to exemplify embodiments and realization of the invention of the present disclosure.

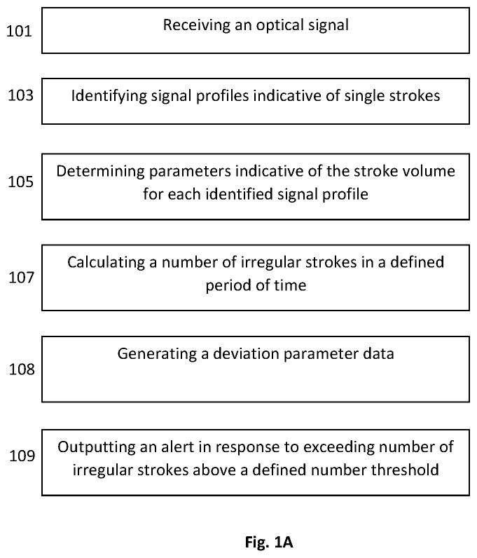

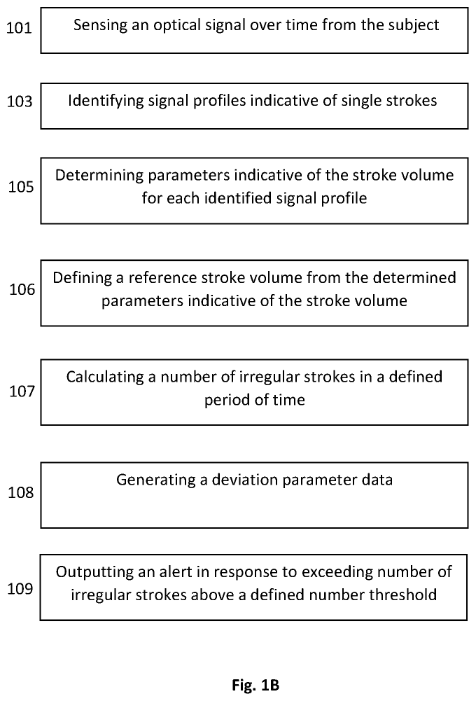

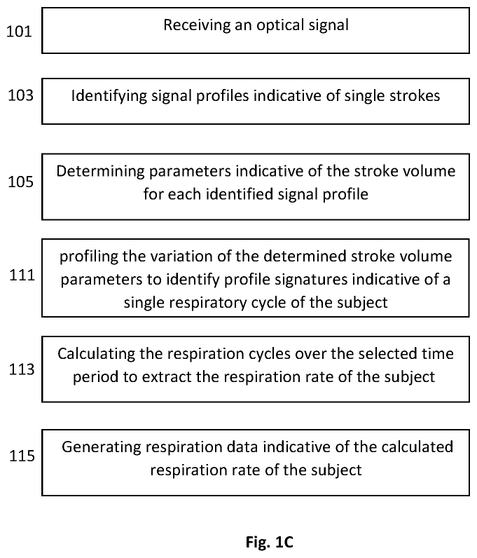

Reference is first being made to FIGS. 1A-1C, which are flow diagrams exemplifying different embodiments of the method according to an aspect of the present disclosure. FIG. 1A shows a first embodiment of the method that includes receiving an optical signal 101 indicative of hear activity of the subject that is collected over a certain period of time, in particular a PPG signal that is collected through a skin portion of the subject. Typically, the signal is continuous or at least quasi-continuous such that it provides a PPG signal of the subject over a certain period of time, or can be a continuous or quasi-continuous signal that is received in real-time. It is to be noted that the received signal can undergo any kind of signal treatment so as to facilitate the following method. The method further includes identifying signal profiles indicative of single strokes 103. The identification is made by analyzing the optical signal and extracting time stamps and/or discernable profile signatures in the optical signal that define single strokes. Following the identification of said signal profiles, a determination of a parameter indicative of the stroke volume 105 is performed. Determining the parameter indicative of the stroke volume can be performed by determining the areas or normalized areas of each signal profile that defines a single stroke, thereby obtaining a series of parameters that are proportional to the stroke volumes identified in the temporal optical signal. The method further includes calculating a number of irregular strokes in a defined period of time 107 in the temporal optical signal. An irregular stroke is defined as a stroke that deviates above a deviation threshold from a reference stroke. Namely, a signal of a healthy patient should exhibit a series of stroke volumes that do not deviate from one another in a significant amount, while a signal of a patient with a cardiac irregularity, such as atrial fibrillation, should exhibit a series of stroke volumes that deviates from one another significantly and their standard deviation is significantly large. It is to be noted that indication of number of irregular strokes can be based on calculation of a standard deviation of all the determined stroke volumes over a certain period of time or based on any other statistical calculation that may indicate a deviation from a standard cardiovascular behavior. The method further comprises generating deviation parameter data indicative of a cardiovascular state of the subject. In this specific, non-limiting example, the deviation parameter data is indicative of the number of irregular strokes volume over a certain period of time. In other examples, the deviation parameter data may carry data on other deviation parameters that can indicate irregularity of cardiovascular parameters of the subject, such as a standard deviation of the stroke volumes. Therefore, in this example, when the number of irregular strokes over a certain period of time exceeds a defined number threshold, the method further includes outputting an alert 109 indicating that the patient has a cardiac irregularity. It should be noted that there are many types of cardiac irregularity that can be identified with a significant deviation of a series of stroke volumes and in order to identify the specific cardiac irregularity, additional measurements may be required, such as an ECG measurement of the patient, even if it is a point ECG measurement, namely a short measurement shortly after the preliminary identification of the cardiac irregularity.

FIG. 1B shows another example of an embodiment of the method according to an aspect of the present disclosure. The example in FIG. 1B differs from that of FIG. 1A by the following: (i) the method includes sensing the optical signal over time from the subject 101 instead of receiving the signal from a measurement that was made not part of the method; and (ii) following the determination of parameters indicative of the stroke volume of each identified signal profile 105, the method further includes defining a reference stroke volume from the determined parameters indicative of the stroke volume of each stroke 106. It is to be noted that there are many ways to define the reference stroke volume, such as defining it as the average volume, the mean volume, the maximal volume, the minimal volume or any other known in the art definition that allows to compare it with the determined stroke volumes to identify cardiac irregularity.

FIG. 1C shows another example of the method according to an aspect of the present disclosure. This example comprises the same steps 101, 103 and 105, as explained with respect to FIG. 1A. After stroke volume parameters are determined 105, the method further comprises profiling the variation of the determined stroke volume parameters to identify profile signatures indicative of a single respiratory cycle of the subject 111. Typically, spontaneous respiratory cycle has a wavy profile of stroke volumes that increases during the cycle to a certain maximum and decreases to a certain minimum. By profiling the variation of the determined stroke volumes, theses minimums and maximums can be identified and therefore a single respiratory cycle can be identified (either between two minimums or between two maximums). The method further comprises calculating the respiration cycles over the selected time period to extract the respiration rate of the subject 113 and generating respiration data indicative of the calculated respiration rate of the subject 115 that can be output or transmitted at any desired form.

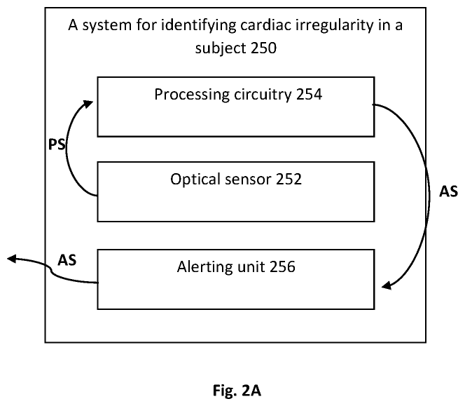

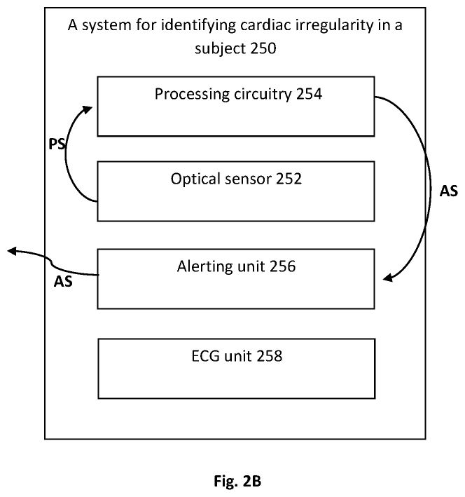

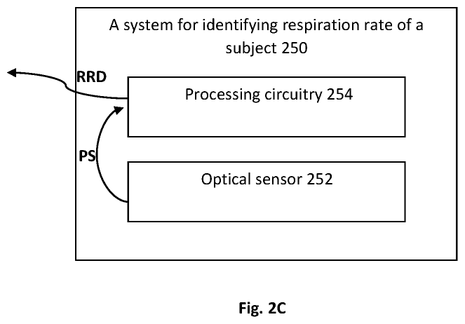

FIGS. 2A-2C are block diagrams exemplifying different embodiments of the system according to an aspect of the present disclosure. FIG. 2A exemplifies a system 250 that is intended for identifying cardiac irregularities in a subject and that includes an optical sensor 252 that is configured to continuously or semi-continuously measuring blood volume change in a tissue or in arterioles under the skin of the subject. Namely, the optical sensor 252 is configured to measure a PPG signal PS over time of the subject and transmit the PPG signal PS to a processing circuitry 254 of the system 250. The processing circuitry 254 is configured to analyze the PPG signal PS to identify time ranges in the signal that define single strokes. It is to be noted that the analysis can include treatment to the signal, such as debugging, smoothing or any pre-process treatment to the signal that is known in the art. For each identified time range, the processing circuitry 254 is configured to extract a parameter indicative of or correlative to the stroke volume of the identified stroke. This can be done, for example, by a certain integration of the signal in the identified time range. The processing circuitry 254 either includes a reference stroke volume in its memory or is configured to generate a reference stroke volume based on a series of extracted stroke volumes. The reference stroke volume can be determined by any mathematical manipulation on the extracted stroke volumes, such as an average value, mean value, maximal value, minimal value, etc. The processing circuitry 254 is further configured to be set with a deviation threshold that defines the allowed deviation of a stroke volume from the reference volume. The processing circuitry 254 is configured to calculate, over a defined period of time, the amount of extracted stroke volumes that exceed that deviation threshold. If over said defined period of time, the processing circuitry 254 counts a number of deviated stroke volumes that is over a defined number threshold, the processing circuitry transmits an alerting signal AS to be executed by an alerting unit 256 of the system 250. The alerting signal AS can be a visual, audible, tactile, vibrational or any other type of signal that indicates the subject or a caregiver of the subject that a cardiac irregularity is identified. It is to be noted that the alerting unit 256 may be part of the processing circuitry 254 or an element separated therefrom.

FIG. 2B exemplifies a system that differs from that of FIG. 2A by also including an ECG unit 258. The ECG unit 258 serves for measuring at least a spot ECG measurement, i.e. a measurement for a defined time window following an identification of a cardiac irregularity. The measurement of the ECG unit 258 is transmitted to the processing circuitry 254 to identify the type of the cardia irregularity. This can be done by matching the signal to known signatures stored in a database and finding the best match of the obtained ECG signal with a stored signature that is indicative of a specific cardia irregularity. Following this process, the processing circuitry 254 can transmit an alert to be executed by the alerting unit 256 indicating the specific identified irregularity.

FIG. 2C exemplifies a system 250 that is intended for determining the respiration rate of a subject and includes an optical sensor 252 that is configured to continuously or semi-continuously measuring blood volume change in a tissue or in arterioles under the skin of the subject. Namely, the optical sensor 252 is configured to measure a PPG signal PS over time of the subject and transmit the PPG signal PS to a processing circuitry 254 of the system 250. The processing circuitry 254 is configured to analyze the PPG signal PS to identify time ranges in the signal that define single strokes. It is to be noted that the analysis can include treatment to the signal, such as debugging, smoothing or any pre-process treatment to the signal that is known in the art. For each identified time range, the processing circuitry 254 is configured to extract a parameter indicative of or correlative to the stroke volume of the identified stroke. This can be done, for example, by a certain integration of the signal in the identified time range. The processing circuitry 254 is further configured for profiling the temporal variation of the determined stroke volumes parameters to identify, in the variation profile, signatures indicative of single respiratory cycle. This can be, for example, identifying two consecutive minima or two consecutive maxima. The processing circuitry is further configured for calculating the number of single respiratory cycles over a selected time window to determine the respiratory rate of the subject. The processing circuitry is configured to generate respiration rate data RRD indicative of the respiration rate of the subject and to allow its output or transmission in any desired form, e.g. for presenting it on a display of a monitor or a watch. It is to be noted that the system that is exemplified in FIG. 2C can be different or the same system as exemplified in FIGS. 2A-2B. If the system is the same, it is capable of performing both respiratory rate determination and cardiac irregularity identification.

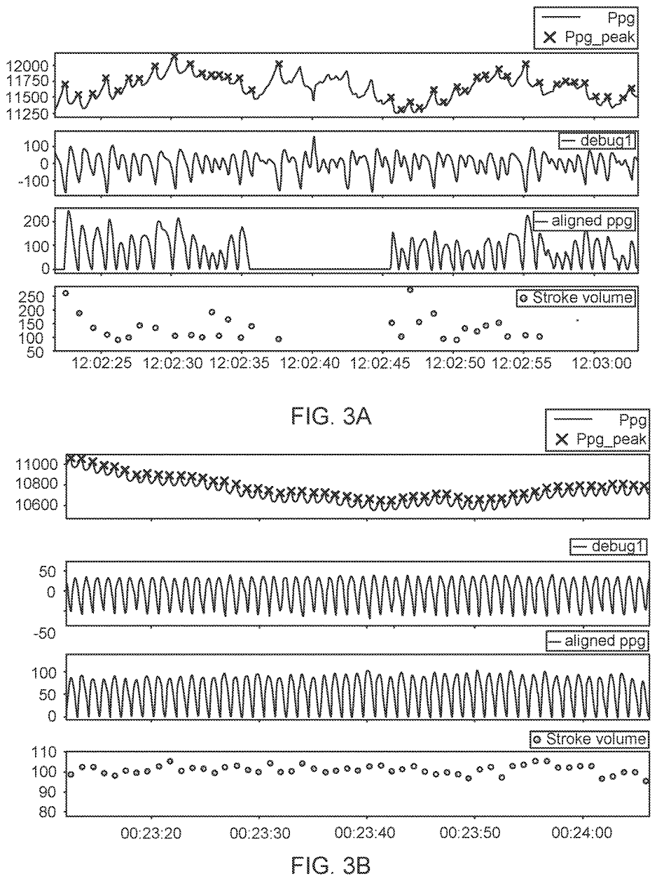

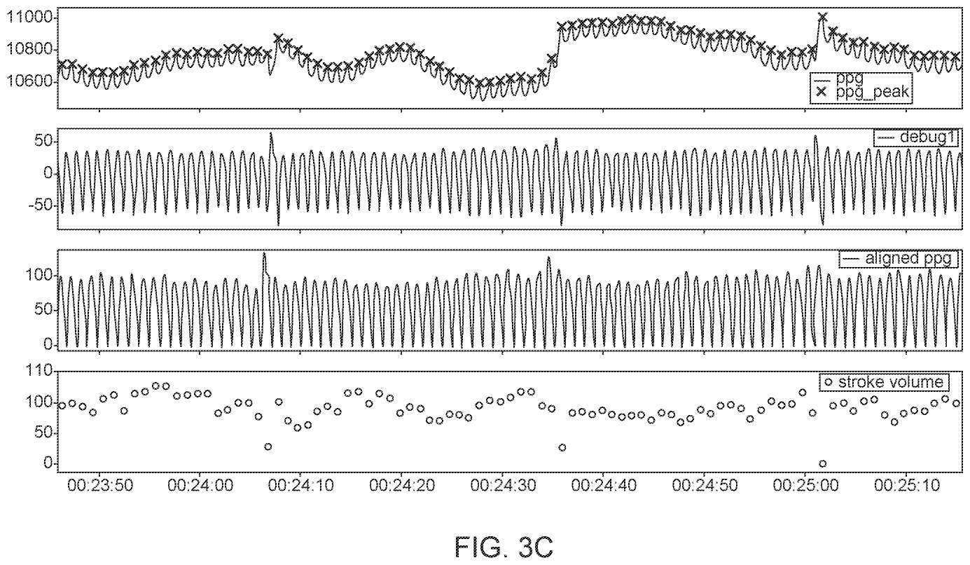

FIGS. 3A-3C are three different examples of PPG signals that were measured over time from three different patients. In each figure, the upper frame shows the signal as measured by the sensor, the frame below it shows a first treatment of the signal, the frame below it shows a second treatment to the signal, in which the signal is aligned to the X axis (namely that each stroke signal starts and ends at a zero value of the Y axis) and the lowest frame shows the values of extracted parameter indicative of the stroke volumes identified in the signal.

FIG. 3A shows an example of a patient that suffers from atrial fibrillation, and it can be appreciated that the standard deviation of the values of the extracted stroke volumes is relatively high.

FIGS. 3B-3C show examples of healthy patients, in which the standard deviation of the values of the extracted stroke volumes is relatively low. In FIG. 3C it can be appreciated that there are some extracted stroke volumes that that exhibit significant deviation, however since they are very random and rare, the processing circuitry identifies them as results of bad signal and not a real deviation of stroke volume.

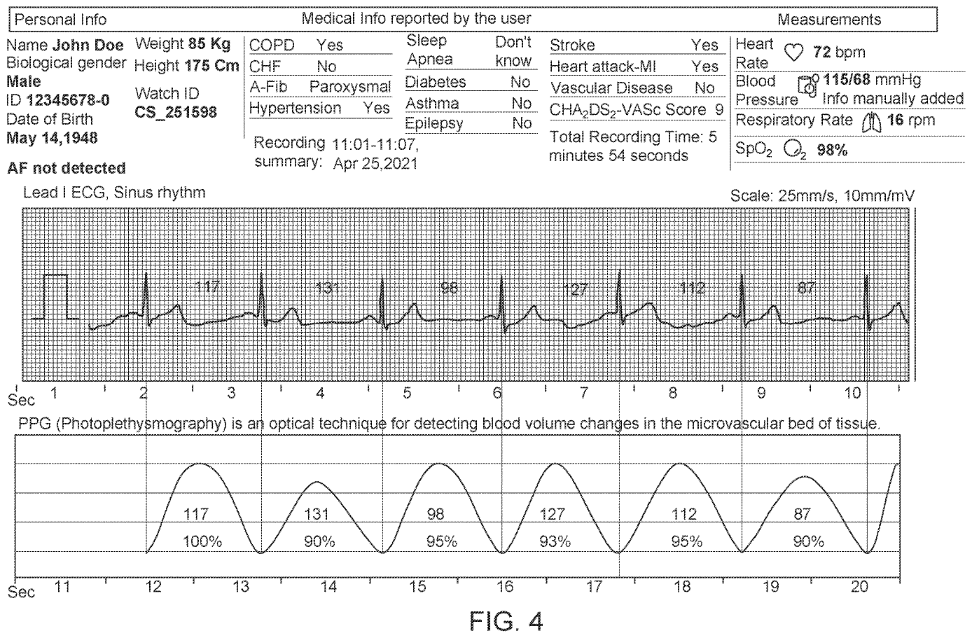

FIG. 4 is an example of a report that can be outputted by the system of the present invention. The report shows the ECG signal of the patient aligned in time with the PPG signal of the patient. The ECG signal can assist the processing circuitry to determine the time ranges in the PPG signal that are associated with each single stroke.

In the report, each pattern that is identified as a single stroke is assigned with a number, in this case percentages from the reference stroke volume to show the deviation of each stroke from the reference stroke volume.

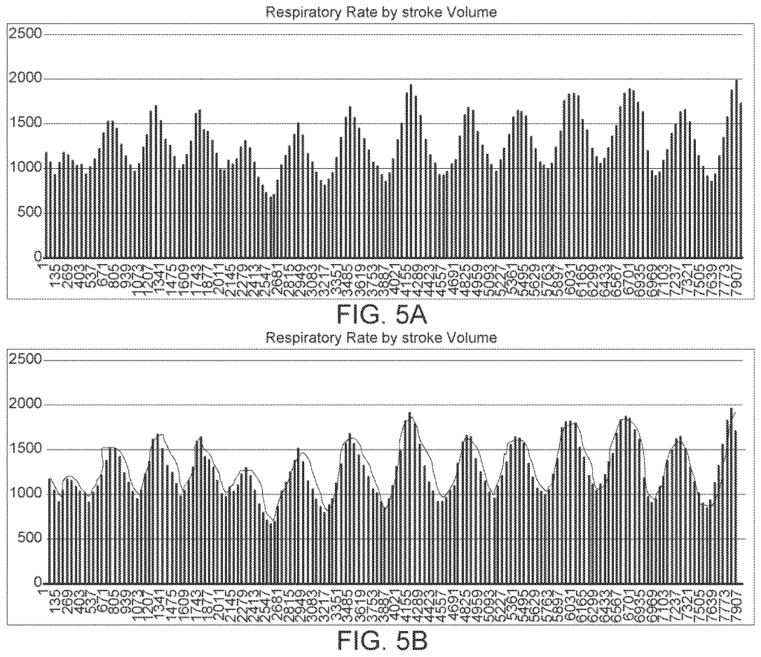

FIGS. 5A-5B show an example of calculated stroke volumes of a subject over time. Each bar in the figures represents the stroke volume extent of the subject. It is to be noted that the bar can only represent a parameter which is proportional to the stroke volume and not the actual stroke volume. FIG. 5B shows a profiling of the variation of the stroke volumes over the period of time of the collected data. The wavy profile is the result of different phases in the respiratory cycle and by identifying consecutive repeating patterns or signatures in the variation profile, a single respiratory cycle can be identified. Then, by calculating the number of single respiratory cycles over a selected time frame in the collected data, the respiratory rate in this time frame can be calculated.

1-53. (canceled)

54. A method for real-time identification of one or more physiological parameters in a subject, comprising:
receiving a temporal optical signal indicative of blood volume changes in the microvascular bed of tissue of the subject, wherein the temporal optical signal is a photoplethysmogram (PPG) signal;
identifying, in one or more selected time periods of said temporal optical signal, signal profiles indicative of single strokes;
based on the optical signal profile, determining a stroke volume parameter indicative of or correlative to the stroke volume of identified single strokes;
wherein the method further comprising at least one of:
(1) for identified single strokes, calculating a deviation parameter, and generating deviation parameter data indicative of said one or more cardiovascular parameters, wherein the deviation parameter data is indicative of the number of irregular strokes volume over a certain period of time, an irregular stroke is defined as a stroke that deviates above a deviation threshold from a reference stroke volume parameter; wherein if the deviation parameter is above a defined deviation parameter threshold, the method comprises generating irregular cardiac activity data based thereon, and outputting said irregular cardiac activity data; and
(2) profiling the variation of the determined stroke volume parameters to identify profile signatures indicative of a single respiratory cycle of the subject, calculating the respiration cycles over the selected time period to extract the respiration rate of the subject, generating respiration data indicative of the calculated respiration rate of the subject, and outputting said respiration data.

55. The method of claim 54, wherein said calculating comprises calculating a number of irregular strokes, irregular strokes being defined by a deviation in their stroke volume parameter that is greater than a defined deviation threshold from the reference stroke volume parameter, wherein the number of irregular strokes is the deviation parameter.

56. The method of claim 54, wherein said calculating comprises comparing identified stroke volume parameters to the reference stroke volume parameter to obtain said deviation parameter; wherein the reference parameter is a subject's reference stroke volume parameter, and the deviation parameter is the number of irregular strokes, wherein irregular strokes are defined by a deviation in their determined stroke volume parameter that is greater than a defined deviation threshold from the subject's reference stroke volume parameter.

57. The method of claim 54, wherein the deviation parameter is indicative of the standard deviation of the stroke volume parameter of identified signal strokes.

58. The method of claim 54, wherein said identifying comprises identifying, in one or more selected time periods of said temporal optical signal, time intervals of single strokes; wherein said determining comprises determining the stroke volume parameter of identified signal strokes in said identified respective time interval.

59. The method of claim 54, wherein said receiving comprises sensing said temporal optical signal from the subject.

60. The method of claim 54, wherein the temporal optical signal is measured from a wrist of the subject.

61. The method of claim 54, wherein said identifying comprises identifying the local minimums of the temporal optical signal and define each two adjacent local minimums as a time interval of a single stroke.

62. The method of claim 54, wherein said identifying comprises identifying local maximums of the temporal optical signal and define the stroke interval as a certain time range that includes time before and after the respective local maximum.

63. The method of claim 54, wherein said determining comprises integrating the signal in each identified time interval, and wherein the integration is performed with respect to a selected base line reference; wherein said base line is defined as the line connecting two local minimums defining said time interval.

64. The method of claim 54, comprising, prior to calculating, comparing each stroke volume parameter to the reference stroke volume parameter to derive a deviation degree of the respective stroke volume parameter from the reference stroke volume parameter.

65. The method of claim 54, wherein the reference stroke volume parameter is either predetermined or determined based on a selected group of the determined stroke volume parameters; wherein the reference stroke volume parameter is determined based on a mean value, average or any combination thereof of said selected group.

66. The method of claim 54, wherein said deviation threshold is defined as a deviation of at least 20%.

67. The method of claim 54, wherein said deviation threshold is defined as a deviation of at least 0.5 of the standard deviation.

68. The method of claim 54, comprising receiving temporal electrocardiogramal of the subject correlated with said temporal optical signal, wherein said identifying comprises initially identifying the time intervals in said temporal ECG signal and using time stamps of the identified intervals in said temporal ECG signal in said temporal optical signal to identify the time interval in said temporal optical signal.

69. The method of claim 54, wherein said alert is one of: visual signal, electronic signal.

70. The method of claim 54, wherein the method comprises only (1) and not (2).

71. The method of claim 54, wherein said profiling comprises identifying two consecutive local minimums and/or two consecutive maximums of the variation profile of determined stroke volume parameters to thereby identify a single respiration cycle.

72. The method of claim 71, wherein the method comprises only (2) and not (1).

73. A system for real-time identification of one or more cardiovascular parameters in a subject, comprising:
at least one processing circuitry configured for:
(i) receiving a temporal optical signal indicative of blood volume changes in the microvascular bed of tissue of the subject, wherein the temporal optical signal is a photoplethysmogram (PPG) signal;
(ii) identifying, in one or more selected time periods of said temporal optical signal, signal profiles indicative of single strokes;
(iii) based on the optical signal's profile, determining a stroke volume parameter indicative of or correlative to the stroke volume of identified single strokes;
(iv) wherein the processing circuitry is further configured for at least one of:
(1) for identified signal strokes, calculating a deviation parameter, and generating deviation parameter data indicative of said one or more cardiovascular parameters; wherein the deviation parameter data is indicative of the number of irregular strokes volume over a certain period of time, an irregular stroke is defined as a stroke that deviates above a deviation threshold from a reference stroke volume parameter; wherein if the deviation parameter is above a defined deviation parameter threshold, the processing circuitry is further configured for generating irregular cardiac activity data based thereon and outputting said irregular cardiac activity data and
(2) profiling the variation of the determined stroke volume parameters to identify profile signatures indicative of single respiratory cycle of the subject, calculating the respiration cycles over the selected time period to extract the respiration rate of the subject, generating respiration data indicative of the calculated respiration rate of the subject, and outputting said respiration data.
